(19) United States
(12) Patent Application Publication
(10) Pub. No.: US 2009/0036794 A1
(43) Pub. Date: Feb. 5, 2009

(54) METHOD AND APPARATUS FOR DETERMINING LOCAL TISSUE IMPEDANCE FOR POSITIONING OF A NEEDLE

(75) Inventors: Audun Stubhaug, Oslo (NO); Orjan Grottem Martinsen, Stabekk (NO); Sverre Joran Grimnes, Oslo (NO)

Correspondence Address: KNOBBE MARTENS OLSON & BEAR LLP, 2040 MAIN STREET, FOURTEENTH FLOOR, IRVINE, CA, 92614, US

(73) Assignee: Rikshospitalet-Radiumhospitalet HF, Oslo (NO)

(21) Appl. No.: 12/159,359

(22) PCT Filed: Dec. 28, 2006
(86) PCT No.: PCT/NO2006/000502

§ 371 Date: Oct. 2, 2008

(30) Foreign Application Priority Data
Dec. 29, 2005 (DK) PA 2005 01845
Aug. 23, 2006 (DK) PA 2006 01096

(51) Int. Cl. A61B 5/053

(52) U.S. Cl. 600/547

(57) ABSTRACT

The invention relates to apparatus and methods for measuring local tissue impedance for subcutaneous tissue surrounding a needle tip inserted into a subject, impedance spectra and/or complex impedance values are determined. The invention applies a monopolar impedance measuring setup with a needle, a current-carrying electrode, an optional reference electrode. The setup is configured to eliminate contributions from the current-carrying electrode in order to measure local impedance of tissue in the close neighbourhood of the needle tip instead of an averaged value over the volume or current path between the needle and the electrode(s). The determined impedance can be correlated with either a tissue type or state, or with a position of the needle tip in the subject, and can thereby provide an insertion history to the operator in the form of impedance or corresponding tissue type as a function of insertion depth or time.

### FIELD OF THE INVENTION

The present invention relates to determining biological tissue types. In particular, the invention relates to methods and apparatuses for determining biological tissue types by measuring electrical impedance values of the biological tissue.

### BACKGROUND OF THE INVENTION

It is often of interest to be able to determine characteristics of the tissue surrounding a probe at locations or under circumstances that do not allow visual inspection.

When administering drugs, tracers or taking biopsies, it is often critical to position the tip of a needle at a specific position or in a specific tissue type. Serious implications and undesired results may incur if the needle unintentionally hits or penetrates veins, arteries, lungs or nerves.

Drugs are often injected intramuscularly without any type of guidance but the experience of the doctor or nurse. Muscular tissue has a large blood flow ensuring fast distribution of the drug. However, if the tip of the needle is positioned in subcutaneous tissue or fatty tissue, localized prolonged high drug concentrations arise that may lead to serious damage, e.g. in the case of steroids. In anesthesia, such as epidural blocks, drugs must be injected near a nerve path or center. Wrong or imprecise injection of the anesthesia results in little or no effect.

Biopsies are carried out by insertion of a needle that can cut and extract a small tissue sample. It may, however, be difficult to ensure that the extracted sample is of the desired tissue type without some form of guidance.

To avoid potential complications, some sort of guidance is used in critical cases. Procedures involving high risks are often carried out under guidance of advanced apparatuses such as X-ray-/CT-, ultrasound-, or MR-imaging. Ultrasound images reveal abrupt changes in acoustical impedance, but are insensitive to homogeneous regions. X-ray images have poor contrast in soft tissues. MRI is sensitive to soft tissue properties, but is complicated to use and requires MRI compatible needles and moving of the patient to a MRI clinic. It is a common disadvantage of the applied guiding technique that the applied equipment is complicated and expensive in relation to the frequent and relatively simple task of inserting a needle with good precision.

In anesthesia, the positioning of the needle is sometimes guided by using the needle to activate the target nerve. By applying an electrically isolated needle (except for the tip) and an electrical signal, the nerve can be stimulated when the tip of the needle is positioned close to the nerve. The activation may result in e.g. flexing of a muscle, which thereby serves as a position feedback.

US 2003/109871 describes an apparatus for detecting and treating tumours using localized impedance measurement. The impedance measurement configuration is described in paragraph [0060] in relation to FIG. 3A. From here it appears that members 22m define sample volumes by means of conductive pathways (22cp) to either between each other or to a common ground electrode (22g or 22gp). It thus appears that the apparatus always measure the impedance of a sample volume of interest (5sv). By switching the electrodes between which the measurement is made, the conductive pathway 22cp is changed which again alters the shape and size of the associated sample volume.

It is a disadvantage of the apparatus described in US 2003/109871 that it measures impedance in relatively large volumes.

Other systems for measuring impedance are described in JP 03272737 and U.S. Pat. No. 6,337,994.

### SUMMARY OF THE INVENTION

It is an object of the present invention to provide apparatus and methods for determining impedance values of tissue surrounding a tip of a needle

The present invention is based on precise determinations of local impedance values in biological tissue surrounding a tip of a needle. Such impedance values allow localized determination of the tissue type and thereby of an anatomical positioning of the needle. Previous attempt to use tissue impedance for positioning have not lead to applicable products. The invention may also be applied in a further characterization of tissue, such as in determining a state of the tissue, e.g. oxygenation, content of substances such as lactic acid. The present inventors are among the world's top experts in electrical bioimpedance, and it is important to realize that tissue impedances are not static well-defined values, but rather trends in relative and/or absolute values. As the state of living tissue may change very fast (e.g. due to excitement, tension or pain of the subject), so may the impedance value. Additionally, the impedance value for the same type of tissue may vary between subjects or between different parts of the same subject, or as a result of ischemia or other pathological conditions. The determination of tissue type through impedance measurements is therefore a challenging task.

In a first embodiment, the invention applies impedance frequency spectra to determine tissue type. In this first embodiment, the invention provides an apparatus for determining a type of tissue surrounding a needle, the apparatus comprising:
an electronic processing unit with an impedance measuring circuit for registering an impedance signal;
a monopolar impedance measuring setup comprising a needle having an electrically conducting first surface part to be inserted into a subject and a current-carrying electrode to be positioned on the skin of the subject, the first surface part and the current-carrying electrode being in electrical connection with the impedance measuring circuit; and
means for providing feedback indicative of a needle position to an operator; the impedance measuring circuit comprising an alternating current or voltage source connected to provide an alternating current or voltage driving signal to the first surface part and to the current-carrying electrode;
   the monopolar impedance measuring setup being configured to at least substantially eliminate impedance contributions from the current-carrying electrode; and
   the electronic processing unit further comprising:
means for varying a frequency of the driving signal from the source;
means for calculating impedance values from the driving signal and the impedance signal for two or more frequencies of the driving signal to form an impedance spectrum;
a memory for holding values corresponding to previously recorded spectral impedance values and corresponding tissue types;
means for determining a tissue type surrounding the first surface part by comparing measured impedance values, or values derived therefrom, with the values from the memory.

Since impedance values are determined using an AC driving signal, different driving signal frequencies yields different impedance values. Throughout the present description, spectral impedance values refer to impedance values at two or more different frequencies, The impedance vs. frequency spectrum Z(f) characterizes the tissue to a much higher degree than single impedance values. The spectrum for different tissue types may be similar in some frequency intervals and very dissimilar in others. Also, impedances in some frequency intervals may be subject to large changes when the state of the tissue changes, while remaining almost unaffected in other frequency intervals. Thereby, the determination of tissue type may be based on one or several segments of the impedance spectrum. This is advantageous since it allows for a much more fine distinction between tissue types under changing conditions.

In a second embodiment, the present invention applies both the modulus and the phase of complex impedance values to determine tissue type. In this second embodiment, the invention provides an apparatus for determining a type of tissue surrounding a needle, the apparatus comprising:
an electronic processing unit with an impedance measuring circuit for registering an impedance signal;
a monopolar impedance measuring setup comprising a needle having an electrically conducting first surface part to be inserted into a subject and a current-carrying electrode to be positioned on the skin of the subject, the first surface part and the current-carrying electrode being in electrical connection with the impedance measuring circuit; and
means for providing feedback indicative of a needle position to an operator;
   the impedance measuring circuit comprising an alternating current or voltage source connected to provide an alternating current (AC) driving signal to the first surface part and to the current-carrying electrode;
   the monopolar impedance measuring setup being configured to at least substantially eliminate impedance contributions from the current-carrying electrode; and
   the electronic processing unit further comprising:
means for calculating complex impedance values having a modulus and a phase from the driving signal and the impedance signal;
a memory for holding values corresponding to previously recorded complex impedance values and corresponding tissue types;
   means for determining a tissue type surrounding the first surface part by comparing both the modulus and the phase of the measured impedance values, or values derived therefrom, with the values from the memory.

A complex number Z can be represented in different ways, such as in an exponential representation, polar representation or Cartesian representation as indicated by the following relations.

$$\begin{matrix}Z = Z {e}^{iφ} \\ = Z ( cos φ + i sin φ ) \\ = R + iX ,\end{matrix}$$

$$φ = arctan \frac{X}{R} , \\ Z = \sqrt{{R}^{2} + {X}^{2}}$$

In dealing with impedances, it is customary to use the exponential or polar representation where Z is the modulus, size or amplitude of the impedance, and φ is the phase difference between the voltage and the current. Hence, the complex impedance provides more characteristics of the tissue than e.g. the impedance values applied in JP 03272737.

Preferably, an apparatus according to the invention applies spectral, complex impedance values, i.e. a combination of the apparatus of the first and second embodiments.

In the present context, tissue means a part of an organism consisting of an aggregate of cells having a similar structure and function. The means for comparing can preferably determine one or more of at least the following tissue types: muscular, fatty, cartilage, connective tissue, epithelia, membranes, epidermis, dermis, parenchyma, body fluids such as spinal fluid, blood, synovia, as well as subcategories within tissues, such as different types of muscular tissue; smooth, striated cardiac, and striated skeletal. Also, position generally refers to an anatomical position in a subject or patient unless otherwise indicated. The anatomical position indicates in which type of tissue or anatomical feature the needle tip is positioned. Although the anatomy is similar for all subjects within a species, e.g. humans, the size of various features (muscles, fatty tissue) may vary to a high degree. The absolute position (e.g. in x,y,z-coordinates) may therefore not reveal which type of tissue or anatomical feature presently surrounds the needle tip.

The first surface part of the needle is the part in contact with the tissue region to be measured upon. The surface area of the first surface part should be relatively small to avoid variation of impedance and/or tissue type over the contact surface, in which case the measurement would reflect an average over the variation. Hence, a surface area of the first surface part is preferably smaller than 15 mm², preferably smaller than 10 mm², such as smaller than 5 mm². This is advantageous in that it allows for a localised determination of the tissue impedance.

The present invention applies the electrical impedance (ratio of voltage to current) to characterise tissue. The person skilled in the art will recognise that the admittance (ratio of current to voltage) may be applied equivalently. In some relations, it is customary to use the term immittance when referring to either the impedance or the admittance of an electrical circuit.

The measured impedance is composed of tissue impedance in series with electrode polarisation impedance. It is known that in some frequency ranges the tissue impedance dominates the measured value, and in other the electrode polarisation impedance dominates. The electrode polarisation impedance is traditionally considered useless and a source of error.

The inventors have realised that the electrode polarisation impedance from the monopolar electrode may dependent on tissue characteristics, and that it may therefore be used to obtain tissue characteristic data. Hence, in a further embodiment, the tissue impedance is determined in a frequency range comprising frequency ranges dominated by polarisation impedance of the first surface part.

The AC driving signal provided by the alternating current or voltage source and the impedance signal measured by the impedance measuring circuit are current and voltage signals from which the impedance is determined. Generally, if the driving signal is generated by an alternating voltage source, the impedance signal is an alternating current signal. Vice versa, if the driving signal is generated by an alternating current source, the impedance signal is an alternating voltage signal. The abbreviation AC generally designates alternating current/voltage signals, and does not determine whether a voltage or a current source provides the diving signal.

The monopolar impedance measuring setup designates the parts having the physical interaction with the subject, primarily the electrodes (the first surface part is an electrode), and refers to e.g. the number of electrodes, their respective size and shape, material composition, dielectric surroundings (e.g. insulated part of needle) etc. That the impedance measuring setup is monopolar means that the measured impedance is due to only one of the electrodes, the needle tip, with negligible contribution from tissue near the other electrodes and between the electrodes. In the embodiments of the invention, the monopolar impedance measuring setup is thereby configured to eliminate or reduce impedance contributions from the current-carrying electrode and any further electrodes. This means that the measured impedance is the local tissue impedance determined only by the tissue in the close proximity of the first surface part of the needle, and not by the entire conducting path or volume between the electrodes used in the measurement.

Systems such as described in JP 03272737 use paired electrodes with an external ring formed reference electrode (8, 17, 37) in contact with the skin. However, the skin is a tissue of very high resistivity so that the impedance of the reference electrode will contribute with an appreciable part of the measured impedance between electrode pairs (7 and 8, 13 and 17, 41 and 37). Skin resistivity is also unstable and very dependent on e.g. sweat level. Accordingly, it becomes impossible to establish a calibrated link between measured impedance values and a tissue type. Only changes in impedance can be determined, as is also indicated in JP 03272737.

In U.S. Pat. No. 6,337,994, the two-part probe consisting of the outer sleeve and the inner stylet with a non-conductive material there between provides a bipolar electrode system, where both the sleeve and the stylet are measuring. The outer sleeve contributes both with electrode polarization and contributions from other tissue regions along the insertion path. This presents no problem when measuring on liquid solutions which are homogeneous so that both outer sleeve and stylet are in the same environment. However, it is expected that the probe will work poorly when applied to inhomogeneous systems containing layers of different tissue, such as in the body on a human or an animal.

In US 2003/109871 it is the impedance of tissue in a sample volume between two or more electrodes which is measured. By switching the electrodes between which the measurement is made, the conductive pathway is changed which again alters the shape and size of the associated sample volume.

The embodiments of the invention solves the above problems of the prior art in different ways. In one embodiment, a measurement localized at the needle tip is ensured by using an additional electrode, a reference electrode, on the skin of the patient and by configuring the impedance measuring circuit to at least substantially eliminate impedance contributions from the reference electrode and the current-carrying electrode. In a preferred implementation, an active operational amplifier circuit is applied, which comprises an operational amplifier having a first input connected to the signal source, a second input connected to the reference electrode and an output connected to the current-carrying electrode. In this monopolar electrode set-up, the current from the AC signal is drawn between the first surface part and the current-carrying electrode, whereas the impedance is measured between the first surface part and the reference electrode. Thereby, the error contribution from the tissue contacting reference electrode and the current-carrying electrode is eliminated—the measured impedance is due only to the tissue surrounding the first surface part of the needle.

In another embodiment, a measurement localized at the needle tip is ensured by using a current carrying electrode which is significantly larger than the area of the first surface part of the needle. The required ratio is dependent on the impedance of the skin, which itself may vary, and the electrode contact material to the skin. In a preferred implementation the size of the current carrying electrode is at least 200 times larger, preferably at least 1000 times larger, than the first surface part.

As will be described in greater detail later, the impedance measuring circuit and setup may be configured so that only tissue within a given distance from the first surface part contributes to the measured tissue impedance values. Hence, it may be preferred that the impedance measuring circuit and setup are configured so that the measured tissue impedance values are substantially determined by tissue within a given distance from the first surface part, the given distance being less than 10 mm, such as less than 8 mm, 5 mm, 3 mm, 2 mm, or 1 mm. By “substantially” is meant that the measured value may depend only very little on tissue not within the given distance, e.g. so that the variation of the measured value as a function of this distant tissue is smaller than the precision required to distinguish between tissue types. Thereby, an unambiguous determination of tissue type within the given distance may be made regardless of the tissue outside the given distance.

The measured impedance values depend on the characteristics of the first surface part of the needle—such as on area, shape, and surface properties, such as roughness, material conductivity etc. Therefore, the measured impedance values are to some degree characteristic for each needle or needle type, and if the previously determined impedance values of certain tissue types are determined using another needle, a needle calibration factor specific for each needle type must be applied when calculating impedance values. It may even be preferred that a needle calibration factor be determined for each needle during an apparatus standardisation procedure. It is preferable that the invention allows for a determination of an impedance value of tissue surrounding the first surface part with an absolute precision better than 2%, or with a relative precision better than 0.5%.

Several methods may be used in the determination of tissue type by comparison of impedance values. In one preferred embodiment, the values corresponding to previously recorded impedance values are principal components determined by multivariate analysis, and the means for determining a tissue type is configured to determine similar principal components for the measured impedance values. Other methods may be applied, such as neural networks.

In a preferred embodiment, the apparatus provides a guiding system aiding a needle operator to a correct positioning of the needle. For this purpose, the apparatus needs to know at which anatomic position, or in which tissue type, the operator want to position the tip of the needle. Hence, in this embodiment the electronic processing unit further comprises means for receiving input from the operator related to a target tissue type. Also, the means for comparing are adapted to notify the operator through the feedback means, if the target tissue type is in contact with the first surface part. The apparatus may thereby also be used as a training or instruction system for teaching operators correct positioning of needles in different applications.

To aid the guiding, the apparatus may further show an insertion history to the needle operator, meaning the tissue types (or impedances) encountered as a function of e.g. time or insertion depth. Such insertion history may be very helpful when an operator has to position the needle in target tissue which is difficult to find, where the needle is withdrawn/advanced repeatedly. For this purpose, the apparatus may further comprise means for determining an insertion depth or means for tracking time during insertion of the needle into the subject, the apparatus being configured to
measure the impedance as a function of insertion depth or time; and
providing an insertion history by displaying impedance or corresponding tissue type as a function of insertion depth or time.

In a preferred embodiment, the means for determining an insertion depth applies a measured capacitive coupling trough an insulated part of the needle, which depends on the insertion depth. This additional feature allows for a linkage between the determined tissue type and a profile of an anatomical model. In order to efficiently illustrate the anatomical position of the needle, the apparatus may further comprise:
means for providing a cross sectional view of a region of the subject, wherein different parts of the view represent different tissue types held by the memory, and
means for indicating, on the means for providing feedback, a position of the needle through the view based on the determined tissue type.
   In practical terms, these features enable showing the position of the needle in an anatomical model of the subject, and thereby constitute a simple but efficient imaging system. The imaging is not an image of the actual needle in the actual subject. Rather, the view is a picture or a graphical representation of a cross-section of the insertion region and the needle is graphical representation of the first surface part.

In a preferred embodiment, the needle is cannulated for fluid administration or extraction, with a distal opening of the needle being adjacent to the first surface part. The first surface part of the needle is preferably located at a distal end part, i.e. at the tip or point of the needle. The needle may be adapted for insertion in tissue in that it comprises a sharply pointed or cutting distal end part. A proximal end part of the needle may be connected to a syringe to allow for fluid administration or extraction. Optionally, the needle may be a biopsy needle.

In a preferred embodiment, the apparatus is portable, such as a handheld apparatus having a volume less than 5 L and a total weight less than 5 Kg.

The invention may be used in methods for performing cosmetic treatment or cosmetic surgery as well as diagnosis for purposes of cosmetic treatment or surgery. Here, the invention aids the positioning of a cannulated needle in a predetermined tissue type in order to administer fluids or particles such as filling, stuffing or colouring substances, typically to dermal and/or epidermal tissue. Alternatively, the cannulated needle may be positioned to extract fluids or particles from the subject. One preferred application being liposuction, in which case the predetermined tissue type is fatty tissue.

Hence, a third embodiment of the invention relates to the cosmetic procedure corresponding to the application of the apparatus according to the first embodiment. The third embodiment provides a method for positioning a cannulated needle in a predetermined type of tissue for cosmetic treatment or surgery using recorded impedance spectra, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
providing data indicative of spectral impedance values corresponding to different tissue types;
placing the current-carrying electrode on a subject;
for at least one position of the needle in the subject:
   A. driving an alternating current or voltage driving signal between the first surface part and the current-carrying electrode;
   B. varying a frequency of the driving signal and measuring impedance signals corresponding to two or more different frequencies in the impedance measuring circuit;
   C. calculating impedance values from the driving signal and the impedance signal for two or more different frequencies of the driving signal; and
   D. comparing calculated impedance values for two or more frequencies, or values derived therefrom, with the provided data to determine a tissue type in contact with the first surface part at the present position;
adjusting the position of the cannulated needle until the predetermined tissue type is in contact with the first surface part.

Similarly, a fourth embodiment of the invention relates to a cosmetic procedure corresponding to the application of the apparatus according to the second embodiment. The fourth embodiment provides another method for positioning a cannulated needle in a predetermined type of tissue for cosmetic treatment or surgery using recorded complex impedance values, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
providing data indicative of complex impedance values corresponding to different tissue types;
placing the current-carrying electrode on a subject;
for a first position of the needle in the subject:
   A. driving an alternating current or voltage driving signal between the first surface part and the current-carrying electrode;
   B. calculating a complex impedance value having a modulus and a phase from the driving signal and the impedance signal;
   C. comparing both the modulus and the phase of the calculated complex impedance value, or values derived therefrom, with the provided data to determine a tissue type in contact with the first surface part at the present position;
adjusting the position of the cannulated needle until the predetermined tissue type is in contact with the first surface part.

The methods according to the following embodiments relate to determining a local tissue impedance in subcutaneous tissue by measuring and interpreting electrical characteristics of tissue. The determined tissue impedance may be used to determine a tissue type or to monitor correct placement of a needle.

A fifth embodiment of the invention relates to methods for determining tissue impedance using recorded impedance spectra. The fifth embodiment provides a method for determining a local tissue impedance of subcutaneous tissue in a subject, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
placing the current-carrying electrode on a subject; and
for a first position of the needle in the subject:
   A. driving an alternating current or voltage driving signal between the first surface part and the current-carrying electrode;
   B. varying a frequency of the driving signal and measuring impedance signals corresponding to two or more different frequencies in the impedance measuring circuit;
   C. calculating impedance values from the driving signal and the impedance signal for two or more different frequencies of the driving signal.

A sixth embodiment of the invention provides another method for determining tissue impedance, here using recorded complex impedance values. The sixth embodiment provides a method for determining a local tissue impedance of subcutaneous tissue in a subject, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
placing the current-carrying electrode on a subject; and
for a first position of the needle in the subject:
   A. driving an alternating current or voltage driving signal between the first surface part and the current-carrying electrode;
   B. calculating a complex impedance value having a modulus and a phase from the driving signal and the impedance signal.

By local tissue impedance is meant the impedance in the proximity of the first surface part, typically the tip of the needle, in contrary to tissue impedance of a volume or a conducting path between two electrodes. In prior art set-ups for measuring tissue impedance, the determined values are averages over inaccurate bounded volumes between the electrodes. The monopolar impedance measuring setup of the present invention is configured to at least substantially eliminate impedance contributions from the current-carrying electrode in the registered impedance signal, so that the signal is characteristic for the volume surrounding the needle tip, regardless of the position of the other electrode(s). This will be demonstrated in greater detail later in relation to FIGS. 6 and 7.

As described previously in relation to the various apparatus', a measurement localized at the needle tip may be ensured by using an additional electrode, a reference electrode, on the skin of the patient and by configuring the impedance measuring circuit to at least substantially eliminate impedance contributions from the reference electrode and the current-carrying electrode. In a preferred implementation, an active operational amplifier circuit is applied, which comprises an operational amplifier having a first input connected to the source, a second input connected to the reference electrode and an output connected to the current-carrying electrode. In this monopolar electrode set-up, the error contribution from the tissue contacting reference electrode and the current-carrying electrode is eliminated—the measured impedance is due only to the tissue surrounding the first surface part of the needle.

The method according to the fifth and sixth embodiments may further comprise moving the needle to a second position and repeating steps for determining a tissue impedance at the second position.

If impedances of different tissue types are known, the calculated tissue impedance may be correlated to a tissue type or a state of the tissue. Hence, the methods may further comprise:
providing data indicative of spectral impedance values corresponding to different tissue types;
comparing calculated impedance values, or values derived therefrom, with the provided data to determine a tissue type in contact with the first surface part at the present position.

As discussed previously, the positioning preferably refers to an anatomical positioning, i.e. positioning in an anatomical features such as a given type of tissue. If impedances of different tissue types are known, and the build up of the region (e.g. order and approximate thickness' of different tissue in the region) is known, the calculated tissue impedance may be correlated to a position. Hence, in a further embodiment, the methods may comprise:
providing a cross sectional view of a region of tissue and data indicative of impedance values corresponding to different parts of the view that represent different tissue types;
comparing the calculated impedance values, or values derived therefrom, with the indicative data corresponding to different parts of the view and indicating the part of the view that represents a tissue type in contact with the first surface part at its present position.

This embodiment solves the problem of correct positioning of a needle, such as positioning at a desired anatomical position. The monitoring or indication of the placement of the needle does not provide therapeutic effects on the subject, and neither does the electrical interaction with the tissue of the underlying measurements. No results or values used in a diagnosis or a treatment is inferred from the positioning or from the underlying measurements. Any diagnosis or decision related to medical treatment lies either distinctly prior to or after the performance of the above methods, and the methods do not require any professional medical evaluation or interaction. Rather, the methods provide optional procedures which may be used by anyone in aiding or guiding the positioning of a needle.

If a target anatomical position is specified, the method may comprise visually or audibly indicating whether the first surface part is positioned in tissue corresponding to the target anatomical position. This may be carried out using a first colour/tone when the first surface part is not positioned in the given type of tissue, and using a second, different colour/tone when the first surface part is positioned in the given type of tissue.

Corresponding to preferred embodiments of the apparatuses according to the first or second embodiments, the methods may further comprise visually indicating the determined tissue type to a needle operator. Also, the methods may comprise determining and applying a needle calibration factor for the calculation of impedance values.

The methods of the fifth and sixth embodiments may be applied in administration of drugs, patient treatment and surgery. In a preferred embodiment, the methods are applied in obtaining a correct positioning of a needle during anaesthetization.

A seventh embodiment applies the methods for determining a tissue type, applying previously described methods for determining tissue type, to provide a method for administering or extracting a fluid in/from a predetermined type of tissue. Here, the following steps are used:
I. positioning a cannulated needle in a subject;
II. determining a tissue type in contact with the needle using the previous methods;
III. adjusting the position of the cannulated needle and repeating step II until the determined tissue type equals the predetermined tissue type; and
IV. administering or extracting fluid through the cannulated needle.

The administered fluids may e.g. be a drug or a dope, anaesthetics, nutritious substances, tracing substance, filling-, stuffing- or colouring substances. Extracted fluids may e.g. be blood samples, biopsies, or fatty tissue.

A further, eighth embodiment relates to apparatuses and/or methods for indicating a state of tissue, such as subcutaneous tissue which cannot easily be visually inspected. The state of the tissue may refer to e.g. oxygenation, cell activity, content of specific substances or other physiological factors which affect the impedance of the tissue. The tenth embodiment applies apparatus of methods similar to the previous embodiments for positioning or determining tissue type, and the features described in relation to these are generally also applicable to the tenth embodiment.

In the seventh embodiment, an impedance measuring circuit connected to a cannulated needle having an electrically conducting first surface part and a current-carrying electrode may be configured to provide an impedance signal when an alternating current or voltage driving signal is driven between the first surface part and the current-carrying electrode; complex and/or spectral impedance values of a region surrounding the first surface part may be calculated from the driving signal and the impedance signal. By comparing the calculated impedance values, or values derived therefrom, with previously recorded spectral and/or complex impedance values corresponding to tissue in different states, a state of the tissue presently surrounding the first surface part may be determined.

The basic idea of the invention is to determine a local impedance value of subcutaneous tissue at a tip of a needle through spectral and/or complex tissue impedance measurements. The determined tissue impedance may be correlated to a tissue type or a state of the tissue, and used to monitor the positioning of a needle.

In the present description, each preferred feature or element described in relation to embodiments of apparatus can also be implemented, where appropriate, in embodiments of methods in proper form, and vice versa. These and other embodiments of the invention will be apparent from and elucidated with reference to the embodiments described hereinafter

### BRIEF DESCRIPTION OF THE DRAWINGS

FIGS. 1A-C are illustrations of basic electronic set-ups according to embodiments of the invention.

FIGS. 2A-C show illustrations of a selection of needle types applicable in the present invention.

FIG. 3 is an illustration of an embodiment of the apparatus for determining tissue type according to the invention.

FIG. 4 is a flowchart illustrating the performance of the electronic processing unit according to an embodiment of the invention

FIG. 5 illustrates measured impedance value variation through layers of different tissues.

FIG. 6 illustrates the setup of a first pilot study examining the size of the measured volume.

FIG. 7 is a graph showing measurements from the first pilot study.

FIGS. 8A-B and 9A-B are graphs showing measured modulus and phase spectra recorded at four different insertion positions in fat (92) and muscle (93) in a pig, for a solid needle (8A-B) and a hollow needle (9A-B).

FIGS. 10A and B are graphs showing measured complex impedance spectra for different tissue types in a pig.

FIG. 11 shows the principal components for different tissue types resulting from a multivariate analysis of measured complex impedance spectra from different tissue types in a pig

### DETAILED DESCRIPTION OF EMBODIMENTS

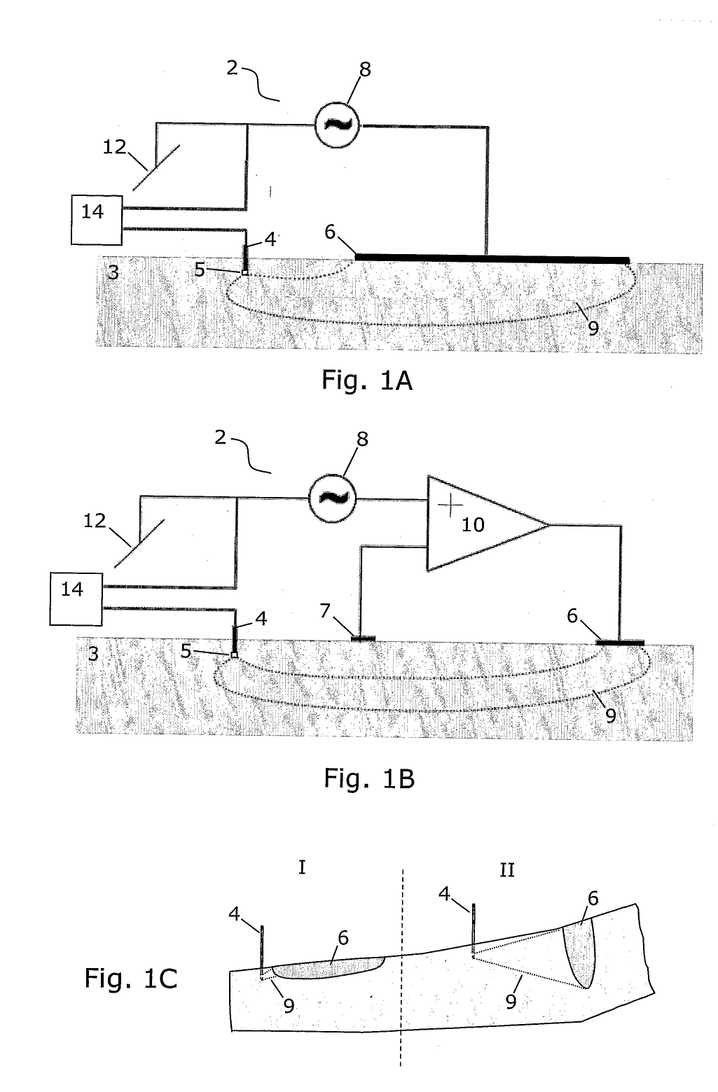

FIGS. 1A and B show different set-ups of the impedance measuring circuit 2 according to embodiments of the invention. In FIG. 1A, cannulated needle 4 with first surface part 5 is positioned in tissue 3. The first surface part 5 is electrically conducting and is connected to the impedance measuring circuit 2, whereas the remainder of the inserted needle surface is either electrically insulating or not connected to the circuit 2. The first surface part, here the tip of the needle, thereby acts as an impedance-measuring electrode. Further, a current-carrying electrode 6, here positioned in contact with the skin of the subject, is connected to the circuit. The circuit comprises an alternating current or voltage supply 8 which is connected to supply an alternating current and voltage signal to the various electrodes. Connected to the impedance measuring circuit 2 is an electronic processing unit 14 receiving the alternating signal (the impedance signal) resulting from the driving signal between the electrodes.

In order for the set-up in FIG. 1A to measure local tissue impedance of the tissue surrounding the needle tip 5, the contribution from tissue in the volume 9 between the needle tip 5 and the electrode 6 should be insignificant. This can be ensured by making the area of the skin electrode (6) significantly larger than the conducting area of the needle tip. The required ratio is dependent on the impedance of the skin, which itself may vary, and the electrode contact material to the skin. However, as a rule of thumb, the size is at least 200 times larger, such as 500 times larger or preferably at least 1000 times larger. Normal EKG electrodes may be used, typically having an area of 2-3 cm². Further, to ensure that a monopolar setup actually used, the relative positions of the point of injection and the current carrying electrode on the skin should be considered. FIG. 1C illustrates two scenarios I and II using the same needle 4 and electrode 6. Due to the orientation and positioning of the needle and electrode in scenario I, the setup is not especially monopolar as only the part of electrode 6 closest to the point of insertion draws current. Instead, electrodes should be positioned as in scenario II when the distance from the needle tip to all points on the electrode is as similar as possible, resulting in a much more cone-shaped volume 9.

In the other embodiment shown in FIG. 1B, the impedance measuring circuit 2 is an active operational amplifier circuit further comprising a reference electrode 7. Using two electrodes on the skin allows for the AC current signal to be drawn between the needle tip and the current-carrying electrode 6, whereas the impedance can be measured between the needle tip 5 and the reference electrode 7. This configuration can thereby eliminate impedance contributions from the reference electrode and the current-carrying electrode, whereby a localized measurement at the needle tip is ensured.

In the embodiment shown in FIG. 1B, an operational amplifier 10 is connected between the current/voltage supply 8 and the current-carrying electrode 6 and reference electrode 7. The operational amplifier 10 has a first input connected to the source 8, a second input connected to the reference electrode 7, and an output connected to the current-carrying electrode 6. In this monopolar electrode set-up, the error contribution from the tissue contacting reference electrode and the current-carrying electrode is eliminated—so that the measured impedance is due only to the tissue surrounding the first surface part of the needle. Again, standard EKG electrodes may be used. In this three electrode configuration, the reference electrode should be closer to the point of injection than the current carrying electrode as illustrated in FIG. 1B. Also, the issues regarding position and orientation made in relation to FIG. 1C in the above in order to ensure a monopolar setup, and thereby a local impedance measurement, are equally valid for this setup.

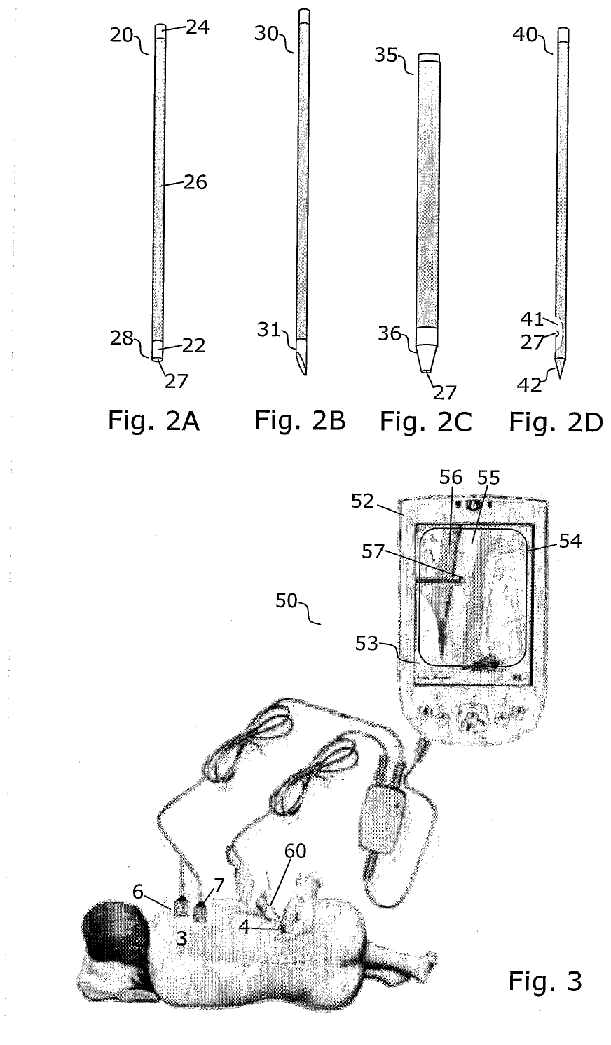

FIG. 2A-C show different needles applicable in the present invention. The needle 20 of FIG. 2A has a first surface part 22 and a terminal part 24 in electrical contact with first surface part 22 for connecting the needle to the impedance measuring circuit 2. The remaining surface part 26 of needle 20 does not have electrical contact to the first surface part 22. The needle 20 is cannulated and has an opening 27 in its distal end 28. Needle 30 of FIG. 2B has a truncated distal end part 31 providing a pointed tip for penetrating skin and/or tissue. Needle 35 of FIG. 2C has a tapered end part 36 ending in opening 27. Needle 40 of FIG. 2D has its first surface part 41 and opening 27 positioned proximal to pointed distal end 42. Typical areas of the first surface parts of applicable needles are in the range 0.1-1 mm².

FIG. 3 shows an illustration of an embodiment of an apparatus 50 for determining a tissue type according to the invention, and for positioning the needle at a given anatomical position. The apparatus comprises the various electrodes (4, 6, 7) and the impedance measuring circuit (not shown) etc. presented previously. The electronic processing unit is embodied by a Personal Digital Assistant (PDA) 52, which also holds a memory for storing impedance values and corresponding tissue types. The PDA can store and execute software for performing the calculations and comparisons according to the invention. The PDA 52 has a graphical interface or screen 53 that can be used to provide feedback indicative of a needle position.

Screen 53 can display a stored cross-sectional view 54 of a region of the subject, also referred to as profiles, in which different parts (55, 56) represent different tissue types. Having determined a tissue type at the location of the first surface part of the needle, the PDA may indicate an anatomic position 57 of the needle in the illustration based on the determined tissue type. It may be preferred that the PDA can store different profiles corresponding to different frequently used points of injection on the human or animal body. Hence, when the operator has to make a difficult insertion, he/she selects the point of injection in a menu, and the apparatus loads a cross-sectional view 54 corresponding to the anatomic profile of the subcutaneous tissue below the point of injection. The anatomic profile may e.g. be a sectional view in through the shoulder or knee region. The apparatus can optionally indicate the exact position of the point of injection as well as an insertion angle for aiding the operator to make an insertion that corresponds to the shown profile.

During insertion, the repeated measurement of local tissue impedance and determination of corresponding tissue type allows the apparatus to indicate to the operator the order in which the various tissue types have been penetrated. Instead of indicating the entire needle as in FIG. 3, it is of interest only to indicate the position of the first surface part (typically the tip). This would also allow the software to indicate on the screen the path or trace of the needle tip during the insertion, the insertion history. It is understood that the path may not be the exact geometrical pathway of the needle tip, but may be the anatomical trace indicating which tissue types has been encountered so far. Often, when trying to position a needle tip in desired tissue in a subject, the operator repeatedly inserts and withdraws the needle until he/she estimates that the needle tip is in the desired tissue. For this purpose, it would be beneficial to indicate the insertion history as tissue type (or impedance) as a function of time or depth, e.g. as a graph with time or insertion depth on the abscissa and different tissue types or measured impedance on the ordinate.

The needle 4 can be operated via a handpiece 60 which can comprise means for determining an insertion depth of the needle into the subject 3 and means for administering or extracting a fluid, such a syringe (not shown).

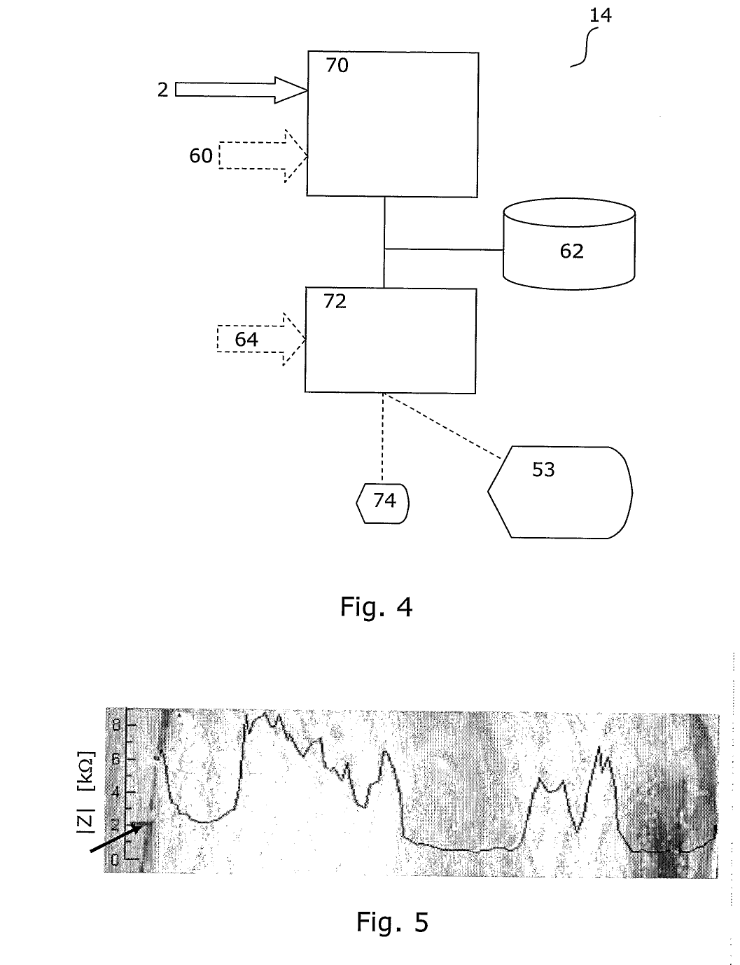

FIG. 4 is a flowchart illustrating the performance of the electronic processing unit 14 of the apparatus 50. Here, the means for calculating an impedance value 70 receives the driving signal and the impedance signal from the impedance measuring circuit 2 and calculates impedance values, e.g. using a needle calibration factor. An insertion depth signal can also be supplied from handpiece 60 so that the impedance values can be calculated as a function of insertion depth. The calculated impedance values are provided to the means for comparing (72) which can also draw data from memory 62. As described previously, the values in memory 62 corresponding to previously recorded impedance values can be principal components determined by multivariate analysis. This will allow determination of (complex) impedance values at different frequencies for a large number of tissue types under controllably varying conditions. The means for comparing can determine similar principal components for the measured impedance values and apply these in the comparison to determine a tissue type.

The determined tissue type can be indicated by indicating an anatomical position of the needle on screen 53 as described in relation to FIG. 3. Optionally, a target tissue type specification 64 can be provided by the operator, in which case means (74) for indicating whether the first surface part is positioned in the target tissue type may be sufficient. Such means 74 can e.g. be red/green diodes, where red indicates that the target tissue type has not been reached and green indicated that it has. Alternatively, the feedback can be given audibly.

FIG. 5 illustrates measured impedance value variation (in kΩ) through different layers of tissues, primarily fatty tissue and muscle. The tissue sample applied here was from dead pig and had been processed for consumption. The black arrow indicates the point of insertion. It can be seen that large variations are measured in the transition between different tissue types, and that the there are statistically significant differences between the measured values in the different tissue types.

To demonstrate the feasibility of the method, results from two pilot studies are summarised in the following. In both studies, the complex impedance measurements were done with a Solartron 1260/1294 system, and two different types of needles 4, solid and hollow, where used as the measuring electrode:
needle a; solid needle, 0.33×37 mm (Medtronic, 9013S0631), first surface part 5 having contact area 0.3 mm²)
needle b: hollow needle 0.7×50 mm (BBraun, Ref: 04894502 “Stimuplex A”)

### The First Study

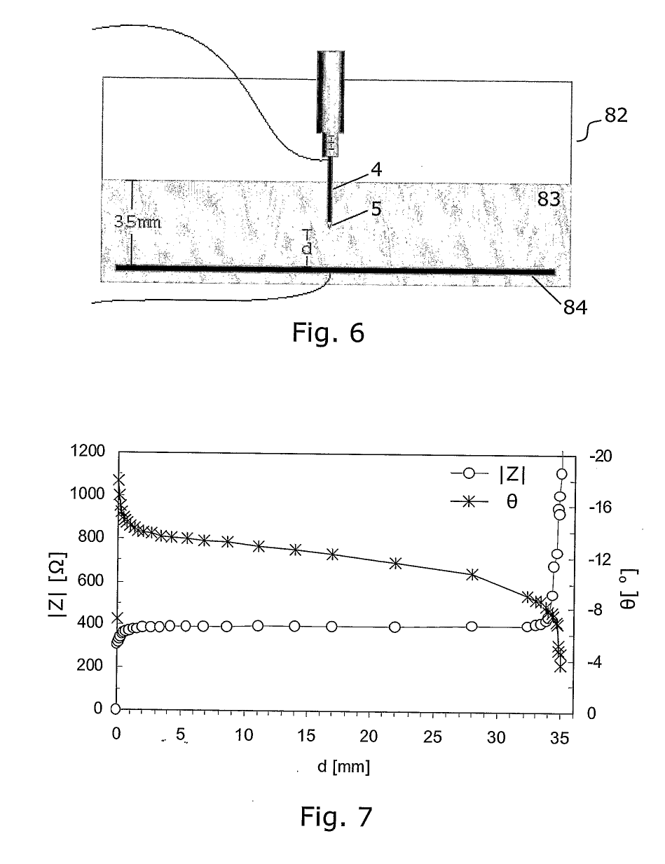

The first study examines the sensitivity zone around the tip of needle a). The needle a), electrode 4 was placed in a saline (0.9% NaCl) filled vessel 82. The vessel had a bottom area of 21×15 cm and was filled to 35 mm height with saline 83. 10.5×15 cm of the vessel bottom was covered with a stainless steel plate 84 used as neutral electrode. The needle position (distance from bottom, d in FIG. 6) was controlled by a micrometer screw, and the needle was moved in small steps in particular near the saline surface and the vessel bottom. The measured complex impedance values at 100 kHz are plotted as a function of d in FIG. 7.

In the setup of FIG. 6, the surface (d=35) represents a boundary to a volume with much higher impedance (air), and the bottom electrode (d=0) represents a boundary to a volume with much lower impedance. Near these boundaries, the measured complex impedance is therefore expected to be influenced by these volumes. From FIG. 7 it can be seen that the modulus |Z| shows only small changes in the interval 3 mm≦d≦32 mm (387Ω−404Ω, Δ|Z|≈40%). In the same interval the phase (0, theta) changes about 30% (13-9 deg.). Both the modulus and the phase changes rapidly as the needle comes within a distance of 3 mm of these boundaries. These results show that about 96% of the measured modulus is due to the area inside a sphere of radius 3 mm surrounding the first surface part. This verifies that with the proper electrode configuration and electronic components, impedance measurements can be use to characterise material in a region closely surrounding the needle tip while reducing contributions from material further away. Thus, the configurations of the impedance measuring circuit and setup in one embodiment are such that only tissue within a given distance from the first surface part contributes to the measured tissue impedance values.

The measured phase angle from d=3 to 32 mm is approximately proportional to the depth of the needle. This effect is mainly originated from the capacitive coupling trough the insulated part of the needle. This capacitance is proportional to the contact area between the electrolyte and the needle, and thereby also proportional to the depth of the needle. For higher frequencies (e.g. 1 MHz) this capacitive coupling will be more pronounced and can be used to determine the insertion depth.

### The Second Study

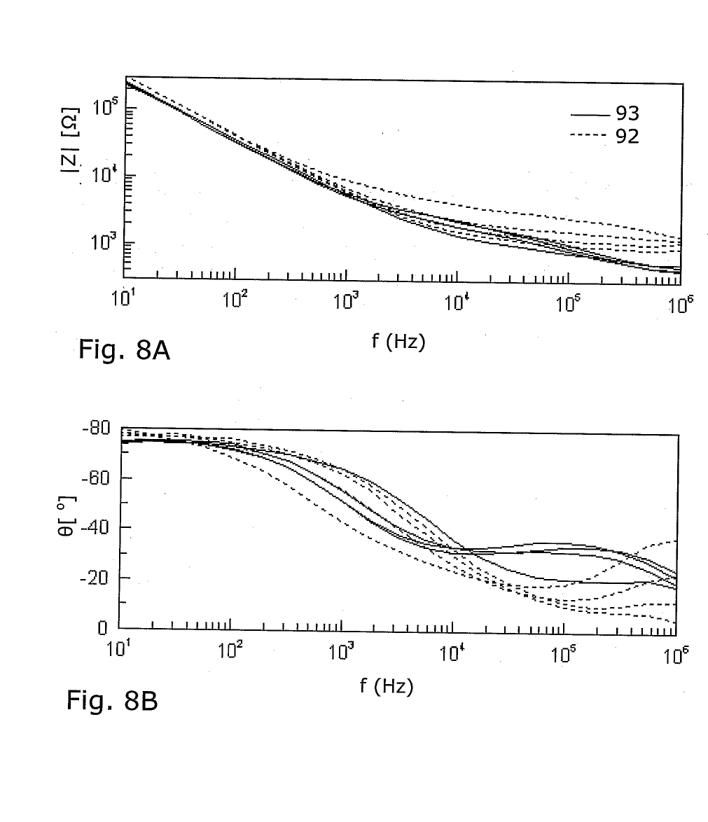

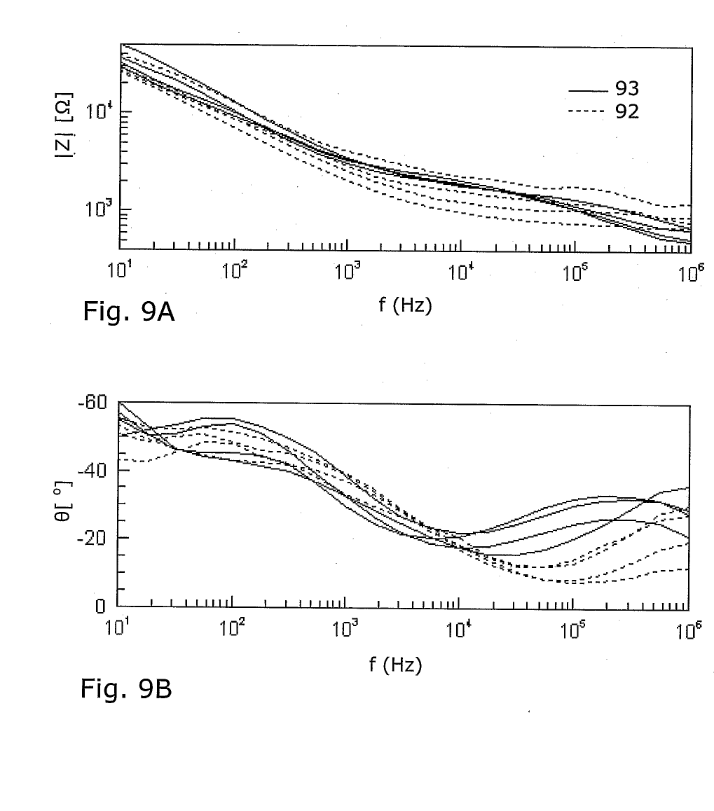

The second study was in-vivo measurements on an anesthetised pig of about 30 kg. As monopolar measuring electrode, solid (needle a) and hollow (needle b) needles were used. Standard ECG-electrodes for reference and current carrying were placed on the skin. The first surface parts of the needles were positioned in different types of tissue. The tissue types were determined by one experienced surgeon and one experienced radiologist through visual inspection and ultrasound imaging. The selection criteria for placement of a first surface part of a needle during measurements were that the surrounding tissue was homogenous. The complex impedance spectrum from 10 Hz to 1 MHz was recorded for each needle position. FIGS. 8A-B, 9A-B and 10A-B show measured modulus (|Z|) and phase (θ) for differences tissue types and needle types.

FIGS. 8A and B shows modulus and phase spectra recorded at four different insertion positions in fat (92, punctured curve) and muscle (93, solid curve) tissue respectively. These measurements were carried out using the solid needle a).

The modulus for fat and muscle are clearly separated in different magnitude ranges above 200 kHz. At frequencies below 300 Hz the data are dominated by electrode polarization impedance, but FIG. 8A shows that this part of the impedance spectra also is tissue dependent. Both these properties can be utilized to distinguish between the two types of tissue. Between 300 Hz and 200 kHz these differences in modulus are not clear, but the phase angle (FIG. 5B) around 30 kHz displays sufficient differences.

FIGS. 9A and B shows modulus and phase spectra recorded at four different positions in fat (92, punctured curve) and muscle (93, solid curve) tissue respectively. These measurements were carried out using the hollow needle b).

The phase angle (FIG. 9B) between 20 and 400 kHz shows characteristic tissue dependent differences, but the separation in modulus (FIG. 9A) is not so obvious for this needle.

A comparison of the low frequency data for the two needles (FIGS. 8A, 8B, 9A and 9B) reveals large differences. At 10 Hz the modulus for needle a) lies between 200 kΩ and 300 kΩ, and between 20 kΩ and 40 kΩ for needle b). The phase angle lies between 70-80 degrees, and 40-60 degrees, respectively. Beside the dependence of tissue type this differences are strongly dependent of size, geometry and material of the needles first surface part. In a preferred embodiment, this dependence can be exploited by the apparatus for an embedded function for automatic detection of the needle type.

In conclusion, for these two types of needle electrodes, it is possible to distinguish between fat and muscle positions by measuring the modulus and phase spectra at frequencies between 10 Hz and 1 MHz.

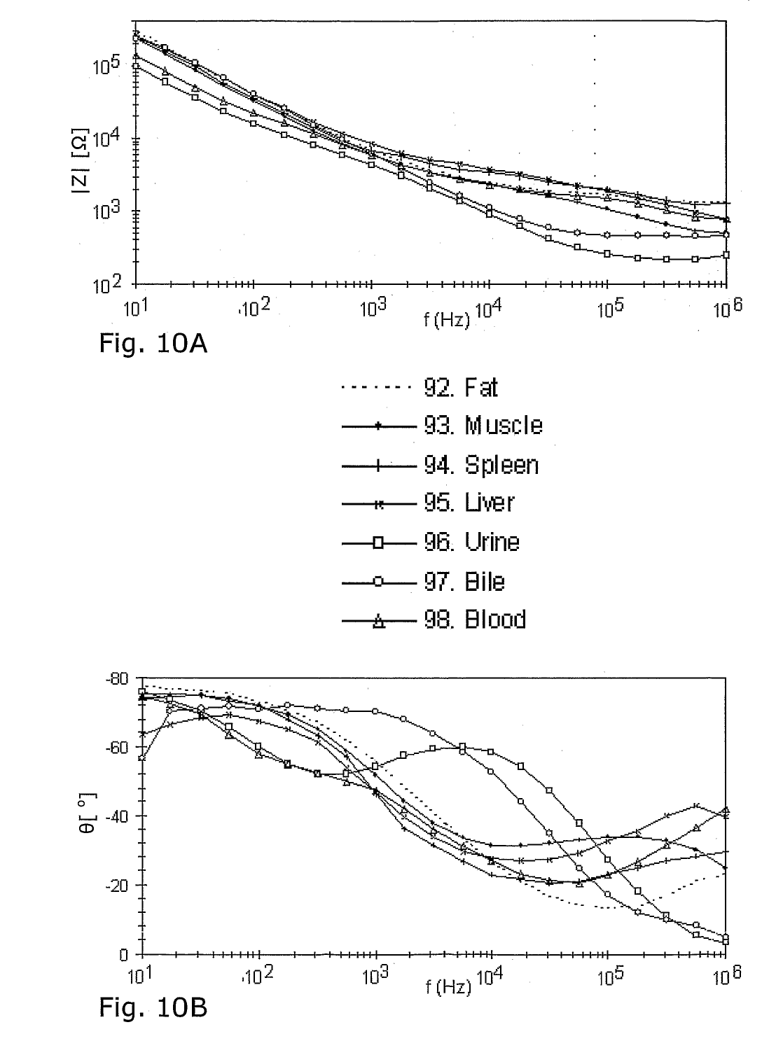

FIGS. 10A and B shows modulus and phase spectra recorded for seven different tissue types with solid needle a. In the figures, curves 92 through 98 show spectra for the different tissue types, where:
92. Fat
93. Muscle
94. Spleen
95. Liver
96. Urine
97. Bile
98. Blood

These measurements show that the complex impedance varies in a manner where the tissue types are distinguishable at some frequencies and not at others. Also, at some frequencies, it is the modulus that differentiates the tissue types whereas at other frequencies, it is the phase information that differentiates the tissue types. The measurements show that complex impedance measurements can be used to clearly distinguish between different tissue types. Hence, measurements of only the modulus |Z| at a single frequency, as presented in the prior art, are generally not sufficient to clearly distinguish between tissue types. Either values for both impedance components are needed (at one or more frequencies), or values of one impedance component for more than one frequency are needed. Preferably, however, frequency spectra for both impedance components are recorded in order to determine tissue type.

In conclusion, the example related to FIGS. 10A and B shows that it is possible to distinguish between spectra recorded in different tissue types by recording of complex impedance spectra and analysing the spectrum pattern over a frequency range.

As is clear from the above, analysis of the complex impedance spectra is not always straightforward, and some complex analysis methods such as multivariate analysis can be applied.

Generally, the tissue type Y of an unknown sample can be calculated from

Y=k₀+k₁·A₁+k₂·A₂+ . . . +kₙ·Aₙ,

where k₀, k₁, k₂, . . . , kₙ are constants previously determined by a regression model, like partial least square (PLS) and/or principal component analysis (PCA), and A₁, A₇, . . . , Aₙ are the measured spectrum parameters from the unknown sample.

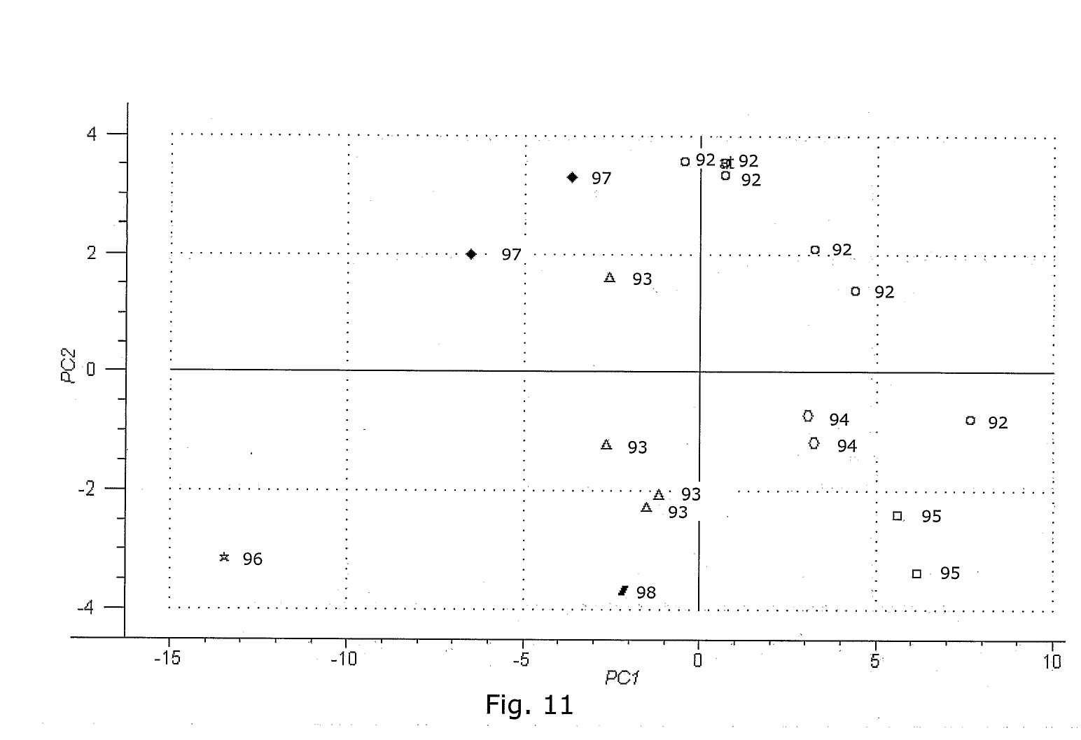

As a specific example, multivariate analysis has been carried out on the data from the second study. 18 different spectra were recorded in a total of seven different tissues, similar to the measurements described in relation to FIGS. 10A and B. The resulting resistance and reactance values were analysed in a multivariate software package (Unscrambler ver. 9.6). The results of the multivariate analysis showing the first two principle components (PC1-PC2) are shown in FIG. 11, using the same denominations as for FIGS. 10A and B.

It is clear from this analysis that spectre belonging to different tissue types are grouped in separate regimes in the PCA diagram. For most of the spectra, the tissue type can be extracted from the position when typical regimes for different tissue types have been mapped out based on laboratory experiments. In conclusion, these results confirms that a tissue type can be determined from a complex impedance spectrum and that, if correlated with a needle position, an anatomical position of a needle can be determined.

1. An apparatus for determining a tissue type of tissue surrounding a needle, the apparatus comprising:
an electronic processing unit with an impedance measuring circuit for registering an impedance signal;
a monopolar impedance measuring setup comprising a needle having an electrically conducting first surface part to be inserted into a subject and a current-carrying electrode to be positioned on the skin of the subject, the first surface part and the current-carrying electrode being in electrical connection with the impedance measuring circuit; and
means for providing feedback indicative of a needle position to an operator;
the impedance measuring circuit comprising an alternating current or voltage source connected to provide an alternating current or voltage driving signal to the first surface part and to the current-carrying electrode;
the monopolar impedance measuring setup being configured to at least substantially eliminate impedance contributions from the current-carrying electrode; and
the electronic processing unit further comprising:
means for varying a frequency of the driving signal from the source;
means for calculating impedance values from the driving signal and the impedance signal for two or more frequencies of the driving signal to form an impedance spectrum;
a memory for holding values corresponding to previously recorded spectral impedance values and corresponding tissue types;
means for determining a tissue type surrounding the first surface part by comparing measured impedance values, or values derived therefrom, with the values from the memory.

2. The apparatus according to claim 1, wherein the impedance values calculated from the impedance signal and the previously recorded impedance values are complex impedance values having a modulus and a phase, and wherein both the modulus and the phase are applied to determine the tissue type.

3. An apparatus for determining a tissue type of tissue surrounding a needle, the apparatus comprising:
an electronic processing unit with an impedance measuring circuit for registering an impedance signal;
a monopolar impedance measuring setup comprising a needle having an electrically conducting first surface part to be inserted into a subject and a current-carrying electrode to be positioned on the skin of the subject, the first surface part and the current-carrying electrode being in electrical connection with the impedance measuring circuit; and
means for providing feedback indicative of a needle position to an operator;
the impedance measuring circuit comprising an alternating current or voltage source connected to provide an alternating current (AC) driving signal to the first surface part and to the current-carrying electrode;
the monopolar impedance measuring setup being configured to at least substantially eliminate impedance contributions from the current-carrying electrode; and
the electronic processing unit further comprising:
means for calculating complex impedance values having a modulus and a phase from the driving signal and the impedance signal;
a memory for holding values corresponding to previously recorded complex impedance values and corresponding tissue types;
means for determining a tissue type surrounding the first surface part by comparing both the modulus and the phase of the measured impedance values, or values derived therefrom, with the values from the memory.

4. The apparatus according to claim 3, wherein the apparatus comprises means for varying a frequency of the AC driving signal from the source, and wherein the means for calculating an impedance value are configured to calculate impedance values for two or more frequencies of the AC driving signal to form an impedance spectrum, and wherein the memory holds, for each tissue type, values corresponding to previously recorded spectral impedance values, and wherein impedance values of two or more frequencies are applied to determine the tissue type.

5. The apparatus according to claim 1, wherein the monopolar impedance measuring setup further comprises a reference electrode to be positioned on the skin of a subject and being in electrical connection with the impedance measuring circuit, and wherein the impedance measuring circuit is configured to at least substantially eliminate impedance contributions from the reference electrode and the current-carrying electrode.

6. The apparatus according to claim 5, wherein the impedance measuring circuit comprises an operational amplifier having a first input connected to the source, a second input connected to the reference electrode and an output connected to the current-carrying electrode.

7. The apparatus according to claim 1, wherein the apparatus can determine an impedance value of tissue surrounding the first surface part with an absolute precision better than 2%.

8. The apparatus according to claim 1, wherein the impedance measuring circuit and setup are configured so that the measured tissue impedance values are substantially determined by tissue within a given distance from the first surface part, the given distance being less that 5 mm.

9. The apparatus according to claim 1, wherein the values corresponding to previously recorded impedance values are principal components determined by multivariate analysis, and wherein the means for determining a tissue type is configured to determine similar principal components for the measured impedance values.

10. The apparatus according to claim 1, wherein the electronic processing unit further comprises means for receiving input from the operator related to a target tissue type, and wherein the means for comparing are configured to notify the operator, through the feedback means, if the target tissue type is in contact with the first surface part.

11. The apparatus according to claim 1, further comprising means for determining an insertion depth of the needle into the subject, and wherein the apparatus is configured to
measure the impedance as a function of insertion depth;
providing an insertion history by displaying impedance or corresponding tissue type as a function of insertion depth.

12. The apparatus according to claim 1, further comprising means for tracking time during insertion of the needle into the subject, and wherein the apparatus is configured to
measure the impedance as a function of time;
providing an insertion history by displaying impedance or corresponding tissue type as a function of time.

13. The apparatus according to claim 1, further comprising:
means for providing a cross sectional view of a region of the subject, wherein different parts of the view represent different tissue types held by the memory, and
means for indicating, on the means for providing feedback, a position of the first surface part in the view based on the determined tissue type,
the apparatus thereby being adapted to indicate an anatomic position of the first surface part.

14. The apparatus according to claim 1, wherein the needle is cannulated for fluid administration or extraction, with a distal opening of the needle being adjacent to the first surface part.

15. The apparatus according to claim 1, wherein the needle is a biopsy needle.

16. A method for positioning a cannulated needle in a predetermined type of tissue for cosmetic treatment or cosmetic surgery, the method comprising the steps of:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
providing data indicative of spectral impedance values corresponding to different tissue types;
placing the current-carrying electrode on a subject;
for at least one position of the needle in the subject:
driving an alternating current or voltage driving signal between the first surface part and the current-carrying electrode;
varying a frequency of the driving signal and measuring impedance signals corresponding to two or more different frequencies in the impedance measuring circuit;
calculating impedance values from the driving signal and the impedance signal for two or more different frequencies of the driving signal; and
comparing calculated impedance values for two or more frequencies, or values derived therefrom, with the provided data to determine a tissue type in contact with the first surface part at the present position;
adjusting the position of the cannulated needle until the predetermined tissue type is in contact with the first surface part.

17. A method for positioning a cannulated needle in a predetermined type of tissue for cosmetic treatment or cosmetic surgery, the method comprising the steps of:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
providing data indicative of complex impedance values corresponding to different tissue types;
placing the current-carrying electrode on a subject;
for a first position of the needle in the subject:
driving an alternating current or voltage driving signal between the first surface part and the current-carrying electrode;
calculating a complex impedance value having a modulus and a phase from the driving signal and the impedance signal;
comparing both the modulus and the phase of the calculated complex impedance value, or values derived therefrom, with the provided data to determine a tissue type in contact with the first surface part at the present position;
adjusting the position of the cannulated needle until the predetermined tissue type is in contact with the first surface part.

18. The method according to claim 16, further comprising the step of administering or extracting fluids or particles through the cannulated needle.

19. The method according to claim 18, wherein administered fluids or particles are filling, stuffing or colouring substances.

20. The method according to claim 18, wherein the cosmetic treatment or surgery is liposuction.

21. A method for determining a local tissue impedance of subcutaneous tissue in a subject, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
placing the current-carrying electrode on a subject; and
for a first position of the needle in the subject:
driving an alternating current or voltage driving signal between the first surface part and the current-carrying electrode;
varying a frequency of the driving signal and measuring impedance signals corresponding to two or more different frequencies in the impedance measuring circuit;
calculating impedance values from the driving signal and the impedance signal for two or more different frequencies of the driving signal.

22. The method according to claim 21, wherein the impedance values calculated from the impedance signal are complex impedance values having a modulus and a phase, and wherein both the modulus and the phase are applied to determine the tissue type.

23. The method according to claim 21, wherein the tissue impedance is determined in a frequency range comprising frequency ranges dominated by polarisation impedance of the first surface part.

24. A method for determining a local tissue impedance of subcutaneous tissue in a subject, the method comprising:
providing a monopolar impedance measuring setup comprising a cannulated needle having an electrically conducting first surface part and a current-carrying electrode, and an impedance measuring circuit being in electrical connection with the monopolar impedance measuring setup for registering an impedance signal, the monopolar impedance measuring setup being configured to at least substantially eliminate impedance contributions from the current-carrying electrode in the registered impedance signal;
placing the current-carrying electrode on a subject; and
for a first position of the needle in the subject:
driving an alternating current or voltage driving signal between the first surface part and the current-carrying electrode;
calculating a complex impedance value having a modulus and a phase from the driving signal and the impedance signal.

25. The method according to claim 24, further comprising:
varying a frequency of the driving signal; and
repeating the steps of measuring and calculating for two or more different frequencies of the driving signal;
wherein the provided data comprises spectral impedance values for each tissue type, and wherein impedance values for two or more frequencies are applied to determine a tissue type.

26. The method according to claim 21, further comprising:
moving the needle to a second position; and
repeating steps A-B(C) for calculating impedance values of tissue type in contact with the first surface part at the second position.

27. The method according to claim 21, further comprising:
providing data indicative of spectral impedance values corresponding to different tissue types;
comparing calculated impedance values, or values derived therefrom, with the provided data to determine a tissue type in contact with the first surface part at the present position.

28. The method according to claim 27, further comprising determining, and indicating to an operator, the tissue type as a function of time or insertion depth.

29. The method according to claim 21, wherein a target anatomical position is specified, and wherein the method further comprises the step of indicating whether the first surface part is positioned in tissue corresponding to the target anatomical position.

30. The method according claim 21, wherein the method further comprises indicating the determined tissue type to a needle operator.

31. The method according to claim 20, further comprising
providing a cross sectional view of a region of tissue and data indicative of impedance values corresponding to different parts of the view that represent different tissue types;
comparing the calculated impedance values, or values derived therefrom, with the indicative data corresponding to different parts of the view and indicating the part of the view that represents a tissue type in contact with the first surface part at its present position.

32. The method according to claim 21, further comprising determining a needle calibration factor, wherein the step of calculating impedance values applies the determined needle calibration factor.

33. A method for administering or extracting fluid in/from a predetermined type of tissue, the method comprising:
positioning a needle in a subject;
determining a tissue type in contact with a first surface part of the needle at the present position using the method according to one of claim 18 or 20, wherein the needle is a cannulated needle;
adjusting the position of the cannulated needle and repeating step II until the determined tissue type equals the predetermined tissue type; and
administering or extracting fluid through the cannulated needle.
